(19) United States
(12) Patent Application Publication
(10) Pub. No.: US 2004/0087602 A1
(43) Pub. Date: May 6, 2004

(54) Stabilized brivudine topical formulations

(76) Inventors: Ulrike Gehlert, Berlin (DE); Karsten Gröger, Berlin (DE); Reinhard Schmitz, Berlin (DE); Karl-Heinz Schrader, Berlin (DE); Andreas Schrader, Berlin (DE); Marc Wihsmann, Berlin (DE); Carlo Alberto Maggi, Pomezia (IT); Stefano Manzini, Pomezia (IT); Bettina Stubinski, Berlin (DE)

Correspondence Address: YOUNG & THOMPSON, 745 SOUTH 23RD STREET 2ND FLOOR, ARLINGTON, VA, 22202

(21) Appl. No.: 10/466,305

(22) PCT Filed: Jan. 10, 2002
(86) PCT No.: PCT/EP02/00163

(30) Foreign Application Priority Data
Jan. 17, 2001 (EP) 01100968.5

(51) Int. Cl. A61K 31/513; A61K 7/025

(52) U.S. Cl. 514/269; 424/64

(57) ABSTRACT

There is disclosed the use of metal-oxides pigments as photodegradation stabilizers in topical compositions containing brivudine.

The present invention refers to stabilized pharmaceutical compositions containing the antiviral Brivudine and their use in the topical treatment of herpesvirus infections. Herpesvirus infections in human can be caused by different herpesviruses, the most common being herpes simplex virus and varicella-zoster virus. There are also many animal herpesviruses.

Herpes simplex virus type 1 causes mucocutaneous infections of the oral cavity which may lead to latent virus persistence and recurrent episodes of herpes labialis (cold sores). Although most herpes labialis episodes are mild, they often cause psychological distress as well as physical discomfort and may be disfiguring in severe cases. As herpes labialis also poses the risk of transmission, there is a need for an effective antiviral treatment of this ailment.

There are a number of antiviral agents, such as acyclovir, famciclovir, penciclovir, brivudine etc., which are active in the treatments of human herpesviruses infections. When localised manifestations of the viral infections occur, a topical treatment may be preferred.

The most active antiviral agents i.e. acyclovir, penciclovir, famciclovir, brivudine etc, all show similar chemical characteristics; in fact their structures resemble that of the purine and pyrimidine bases which constitute the nucleic acids. The antiviral activity of these nucleoside analogues is based on the inhibition of viral DNA synthesis. They are phosphorylated in the infected cell by virus encoded enzymes and they either are inserted as “false blocks” into viral DNA or inhibit the viral DNA directly.

Brivudine, (E)-5-(2-bromovinyl)-2′-deoxyuridine, is a potent antiviral agent for the treatment of varicella zoster virus (VZV) and herpes simplex type 1 virus (HSV-1) infections. Its in vitro efficacy against VZV is superior to both acyclovir and penciclovir [mean IC₅₀ in 13 clinical VZV strains: 0.001 μg/ml (brivudine), 0.2 μg/ml (acyclovir), and 0.91 μg/ml (penciclovir)] (Andrei G., Snoeck R., Reymen D. Eur. J Clin. Microbiol. Infect.; 14; 318-319; 1995).

Furthermore, brivudine is a potent inhibitor of HSV-1 replication with a superior in vitro efficacy compared to acyclovir and penciclovir: in a panel of 23 clinical HSV-1 strains, brivudine proved to be almost twice as effective as acyclovir [mean IC₅₀: 0.52 μg/ml (brivudine) vs. 0.92 μg/ml (acyclovir)] (Andrei G., Snoeck R., Goubau P. Eur. J. Clin. Microbiol. Infect.; 11; 143-151; 1992). In two similar assays in a panel of 20 clinical isolates penciclovir showed a mean IC₅₀ of 0.6 μg/ml and 0.8 μg/ml (Weinberg A., Bate B. J., Masters H. B. Antimicrob. Agents Chemother.; 36; 2037-2038; 1992).

Earlier investigations predominantly in HSV-1 laboratory strains demonstrate an even lower IC₅₀ of brivudine between 0.004 μg/ml and 0.2 μg/ml (depending on the viral strain and the cell system) (De Clercq E., Descamps J., Verhelst G., J. Infect. Dis.; 141; 563-574; 1980; De Clercq E., Antimicrob. Agents Chemother.; 21; 661-663; 1982).

Several patents teach how to prepare a pharmaceutical composition containing an antiviral agent:

GB1,523,865 describes several examples of pharmaceutical compositions useful for administering acyclovir via different routes of administrations: oral, parenteral, ocular or topical route;

EP44543 refers in particular to the cutaneous route of administration and describes several acyclovir formulations suitable for skin application as aqueous creams or in particular oil/water emulsions;

EP948332 discloses pharmaceutical compositions suitable for administration by means of a topical applicator containing acyclovir alone or with vitamin A for the treatment of herpes labialis;

EP809498 discloses pharmaceutical compositions containing an antiviral agent, among which acyclovir or Brivudine, in association with an anti-inflammatory glucocorticoid;

DE3706421 discloses pharmaceutical compositions for the topical treatment of herpesvirus containing 5-ethyl-2′desoxyuridine and urea;

EP72137 refers in particular to derivatives of 3-methyl-5-halovinyl-deoxyurine in the treatment of herpes simplex or varicella-zoster;

EP104066, EP95292, EP95294 disclose derivatives of 2′-deoxyuridine for the topical and systemic treatment of viruses;

GB1601020 first claims brivudine, its synthesis and its use in the treatment of herpes simplex via different routes.

In no case the information available from known pharmaceutical compositions is helpful for preparing stable pharmaceutical composition containing brivudine for topical administration.

Brivudine, in fact, has revealed particularly unstable in the conditions normally used for topical preparations. In those conditions brivudine was found to undergo an extensive photodegradation, which reduces the concentration of the active principle, decreasing the ultimate effect of the treatment and determining a significant local irritation that can discourage from using the pharmaceutical composition. The degradation of brivudine was investigated according to the ICH (International Conference on Harmonization) guideline CPMP/ICH/279/95. Unpacked samples of brivudine, which do not contain any stabilizer, were irradiated for 9.6 hours at an intensity of 49.5 W/m² (dose: 475 Wh/m²). The brivudine content decreases from about 100% to about 70% after irradiation. At the same time the samples show an increase of known and unknown impurities of about 30%.

A suitable photo-stabilizer, in addition to preventing the degradation of the active substance, should possess important characteristics, like absence of toxicity in the range of concentrations used, no undesired biological activities, low cost, physico-chemical properties suitable for industrial handling, no chemical interaction with the other ingredients of the formulation.

Many photo-stabilisers showed ineffective in stabilising topical preparation of brivudine. Furthermore, the knowledge deriving from formulations of antiviral agents similar to brivudine, proved of no help in solving the problem of photodegradation of brivudine in topical pharmaceutical compositions.

It has been surprisingly discovered that the same problem can be solved using suitable amounts of pigments of the metal-oxides type.

Studies of photo-stabilization were conducted encapsulating the active ingredient in cyclodextrin-complexes (e.g. α-, β-, γ-cyclodextrin-complexes) or adding different stabilizers. Known photo-stabilizers like anti-oxidants (e.g. propylgallate, alfa-liponic acid), chemical UV-filters (e.g. octyl triazone) and pigments (titanium dioxide, zinc oxid, iron oxid yellow) were used.

The efficiency of photo-stabilization was tested by determination of the content of brivudine after 2 hours of irradiation with UV-lamp (dose of 1 MED [Minimal Erythimal dose, 24.8 Wh/m², UVA-light]. UVA-light was applied directly to the cream without primary packaging The higher the content of unchanged brivudine after irradiation, the better the photo-stability.

Without photo-stabilizer the brivudine content decreased, after irradiation, to about 40% of the initial concentration (100% corresponds to a concentration of 5% w/w of brivudine in the tested formulation). Encapsulation of brivudine in cyclodextrin-complexes, the addition of anti-oxidants (propylgallate, 0.02% w/w) or chemical UV-filter (octyl triazone, 5% w/w) resulted in a brivudine content between 30% and 60% of the initial concentration. Better results could be surprisingly obtained with pigments such as titanium dioxide, iron oxide or zinc oxide. Titanium dioxide at a concentration of 20% w/w has resulted in about 85% brivudine final content. The pigment iron oxide yellow at a concentration 20% w/w also gave about 85% brivudine content.

From these results it can be concluded that photo-stabilizers of the metal-oxide type are significantly more effective in preventing brivudine photodegradation.

Accordingly, object of the invention are brivudine topical compositions stabilized against photo-degradation by means of pigments of the metal-oxide type. Examples of suitable metal oxides are titanium dioxide, iron oxide and zinc oxide. The pigments titanium dioxide and iron oxide are preferred. Titanium dioxide has a white colour, while iron oxide yellow leads to a yellow coloured cream. Iron oxide can be successfully used also as a colouring agent for the titanium-containing white creams. Such pigments may be used alone or in combination.

Pigment concentrations able to produce an effect of photo-stabilization can vary from 10 to 50% by weight of the composition, preferably from 15 to 35%, more preferably from 20 to 30% w/w. The content of brivudine usually ranges from 0.3 to 8%, preferably from 0.5 to 5% w/w.

A chorioallantoic membrane test on hen's eggs (HET/CAM test) was performed to determine the irritation potential of a formulation with commercial titanium dioxide (25% w/w). The test was carried out after two hours of irradiation with an UV-lamp at 5 MED (124 Wh/m², UVA-light). UVA-light was applied directly to the cream without primary packaging. In these tolerability tests, the cream without the pigment scored “moderately irritant”, whereas that containing the pigment scored “slightly irritant”. The results surprisingly demonstrate that titanium dioxide not only increases photo-stability, but also decreases the irritation potential.

According to the invention, preferred topical compositions are in form of O/W or W/O creams, lipogel, hydrogel or lipstick.

In general, pharmaceutical compositions containing brivudine for topical administration can be prepared according to known methods, for example as described in Remington's Pharmaceutical Science, 17^{th} ed., Mack Publishing Company, Easton, Pa. (1985). By way of example, the O/W cream can be prepared following a three steps procedure which comprises emulsification, addition of the pigment, and addition of the active ingredient.

### EXAMPLES

### Example 1

### O/W cream

| | Quantity [g] |
|---|---|
| Ingredient | per 100 g |
| Brivudine | 1 |
| Titanium dioxide | 25 |
| Aluminium oxide | 1.6 |
| Silica | 0.1 |
| Glycerol | 0.4 |
| Simethicone | 0.5 |
| Paraffin oil, subliqu. | 5 |
| Propylene glycol | 15 |
| Vasilinum album | 9 |
| Polyoxyethylene-monostearate | 2 |
| Cetostearyl alcohol | 1.5 |
| Citric acid 20% | 0.6 |
| 4-Methylhydroxybenzoate | 0.15 |
| Benzalkonium chloride | 0.2 |
| Polydimethylsiloxane | 0.2 |
| Purified water | ad. 100 |
| Sum | 100 |

The excipients forming the oil phase (paraffin oil, subliqu., polyoxy-ethylenemonostearate, cetostearyl alcohol, vasilinum album, polydimethylsiloxane, simethicone) are molten at a temperature between 75° C. and 80° C. The hydrophilic cream base (citric acid 20%, purified water, propylene glycol [partial amount], benzalkonium chloride, 4-methylhydroxybenzoate) is heated separately to a temperature between 75° C. and 80° C. Both phases are combined within a preheated container of a pharmaceutical homogenizer. The mixture is then stirred and homogenized at a temperature between 75° C. and 80° C. and at low pressure. Then the excipients titanium dioxide, aluminium oxide, glycerol and silica are added to the cream base. This blend is stirred and homogenized at a temperature between 75° C. and 80° C. and low pressure. Then the cream is cooled down to room temperature maintaining stirring and low pressure.

Brivudine is dispersed in propylene glycol (remaining amount). This dispersion is added to the cream. The blend is stirred and homogenized at a temperature between 20° C. and 30° C. and at low pressure.

The cream is stored protected from light in closed containers until further processing.

### Example 2

### Lipstick Formulation

| | Quantity [g] |
|---|---|
| Ingredient | per 100 g |
| Brivudine | 5 |
| Titanium dioxide | 20 |
| Glycerol monostearate | 10 |
| Glycerol ester of fatty acids C10-C18 | 20 |
| Propylene glycol | 15 |
| Caprylic acid triglycerid | 15 |
| Capric acid triglycerid | |
| Cera alba | 4 |
| Ester of diglycerol and caprylic-, | 11 |
| capric-, isostearate-, adipic acid | |
| Sum | 100 |

### Example 3

### Lipogel

| | Quantity [g] |
|---|---|
| Ingredient | per 100 g |
| Brivudine | 5 |
| Paraffinum perliquidum | 34 |
| Isopropyl myristate | 25 |
| Silica | 6.25 |
| Sorbitan Monooleate | 3 |
| Titanium dioxide | 16.25 |
| Aluminum oxide | 5.125 |
| Iron oxide yellow | 2.1 |
| Iron oxide red | 0.6 |
| Iron oxide black | 0.3 |
| Glycerol | 2.375 |
| Sum | 100 |

### Example 4

### W/O-Emulsion

| | Quantity [g] |
|---|---|
| Ingredient | per 100 g |
| Brivudine | 0.5 |
| Zink oxide | 10 |
| Titanium dioxide | 10 |
| Paraffinum subliquidum | 15 |
| Propylene glycol | 8 |
| Vasilinum album | 6 |
| Lanolin | 5 |
| Sorbitan monooleate | 3 |
| Cera alba | 1.5 |
| Zinc stearate | 1.0 |
| Magnesium stearate | 1.0 |
| 4-Methylhydroxybenzoate | 0.15 |
| Benzalkonium chloride | 0.2 |
| Propyl gallate | 0.02 |
| Citric acid 20% | 0.6 |
| Purified water | ad. 100 |
| Sum | 100 |

### Example 5

### Hydrogel

| | Quantity [g] |
|---|---|
| Ingredient | per 100 g |
| Brivudine | 5 |
| Titanium dioxide | 21.25 |
| Aluminum oxide | 1.25 |
| Silica | 0.125 |
| Glycerol | 2.375 |
| Iron oxide yellow | 3.25 |
| Iron oxide red | 1.25 |
| Iron oxide black | 0.5 |
| Propylene glycol | 20 |
| Paraffinum subliquidum | 5 |
| Isopropyl myristate | 5 |
| Cetyl alcohol | 3 |
| Polyoxyethylene-monostearate | 0.8 |
| Hydroxyethylcellulose | 0.3 |
| Citric acid | q.s. |
| Purified water | ad. 100 |
| Sum | 100 |

### Example 6

### W/O-Emulsion

| | Quantity [g] |
|---|---|
| Ingredient | per 100 g |
| Brivudine | 5 |
| Titanium dioxide | 22.5 |
| Paraffinum subliquidum | 15 |
| Propylene glycol | 10.5 |
| Vasilinum album | 6 |
| Lanolin | 5 |
| Sorbitan monooleate | 3 |
| Cera alba | 1.5 |
| Zinc stearate | 1 |
| Magnesium stearate | 1 |
| 4-Methylhydroxybenzoate | 0.15 |
| Benzalkonium chloride | 0.2 |
| Propyl gallate | 0.02 |
| Purified water | ad. 100 |
| Sum | 100 |

### Example 7

### Hydrogel:

| | Quantity [g] |
|---|---|
| Ingredient | per 100 g |
| Brivudine | 1 |
| Titanium dioxide | 27 |
| Propylene glycol | 15 |
| Paraffinum subliquidum | 5 |
| Isopropyl myristate | 5 |
| Cetyl alcohol | 3 |
| Polyoxyethylene-monostearate | 0.8 |
| Hydroxyethylcellulose | 0.3 |
| 4-Methylhydroxybenzoate | 0.15 |
| Benzalkonium chloride | 0.2 |
| Citric acid | q.s. |
| Purified water | ad. 100 |
| Sum | 100 |

### Example 8

### Lipogel

| | Quantity [g] |
|---|---|
| Ingredient | per 100 g |
| Brivudine | 2 |
| Iron oxide yellow | 0.75 |
| Iron oxide red | 0.15 |
| Iron oxide black | 0.1 |
| Isopropyl myristate | 24 |
| Titanium dioxide | 15 |
| Paraffinum subliquidum | 15 |
| Propylene glycol | 15 |
| Vasilinum album | 12 |
| Zinc oxide | 5 |
| Silica | 5 |
| Sorbitan trioleate | 3 |
| Cera alba | 3 |
| Sum | 100 |

### Example 9

### O/W Cream

| | Quantity [g] |
|---|---|
| Ingredient | per 100 g |
| Brivudine | 5 |
| Zinc oxide | 10 |
| Titanium dioxide | 10 |
| Aluminium oxide | 0.6 |
| Silica | 0.032 |
| Glycerol | 0.1 |
| Iron oxide yellow | 2.6 |
| Iron oxide red | 1 |
| Iron oxide black | 0.4 |
| Propylene glycol | 20 |
| Vasilinum album | 10 |
| Paraffin oil, subliqu. | 6 |
| Polyoxyethylene-monostearate | 3 |
| Cetostearyl alcohol | 2 |
| Citric acid 20% | 0.6 |
| Polydimethylsiloxane | 0.5 |
| Purified water | ad. 100 |
| Sum | 100 |

### Example 10

### O/W Cream

| | Quantity [g] |
|---|---|
| Ingredient | per 100 g |
| Brivudine | 2 |
| Titanium dioxide | 21.27 |
| Aluminium oxide | 1.38 |
| Silica | 0.07 |
| Glycerol | 0.28 |
| Iron oxide yellow | 1.4 |
| Iron oxide red | 0.4 |
| Iron oxide black | 0.2 |
| Propylene glycol | 20 |
| Vasilinum album | 9 |
| Paraffin oil, subliqu. | 5 |
| Polyoxyethylene-monostearate | 3 |
| Cetostearyl alcohol | 2 |
| Citric acid 20% | 0.8 |
| Polydimethylsiloxane | 0.3 |
| Purified water | ad. 100 |
| Sum | 100 |

The excipients forming the oil phase (paraffin oil, subliqu., polyoxy-ethylenemonostearate, cetostearyl alcohol, vasilinum album, polydimethylsiloxane) are molten at a temperature between 75° C. and 80° C. The hydrophilic cream base (citric acid 20%, purified water, propylene glycol [partial amount]) is heated separately to a temperature between 75° C. and 80° C. Both phases are combined within a preheated container of a pharmaceutical homogenizer. The mixture is then stirred and homogenized at a temperature between 75° C. and 80° C. and at low pressure. Then the excipients titanium dioxide, iron oxide yellow, iron oxide black, iron oxide red, aluminium oxide, glycerol and silica are added to the cream base. This blend is stirred and homogenized at a temperature between 75° C. and 80° C. and low pressure. Then the cream is cooled down to room temperature maintaining stirring and low pressure.

Brivudine is dispersed in propylene glycol (remaining amount). This dispersion is added to the cream. The blend is stirred and homogenized at a temperature between 20° C. and 30° C. and at low pressure.

The cream is stored protected from light in closed containers until further processing.

1. A stabilized topical pharmaceutical composition containing brivudine ((E)-5-(2-bromovinyl)-2′-deoxyuridine) as the active substance and one or more metal-oxide pigments in a concentration from 10% to 50% w/w, together with pharmaceutically acceptable excipients.

2. A pharmaceutical composition according to claim 1 wherein the metal-oxide concentration is from 15% to 35% w/w.

3. A pharmaceutical composition according to claim 2 wherein said concentration is from 20% to 30% w/w.

4. A pharmaceutical composition according to any preceding claims, wherein the pigment is selected from the group consisting of titanium dioxide, iron oxide, zinc oxide.

5. A pharmaceutical composition according to claim 4, wherein the iron oxide is yellow, red or black.

6. A pharmaceutical composition according to claims 4-5, wherein the pigment is titanium dioxide or iron oxide yellow.

7. A pharmaceutical composition according to any preceding claim, containing 0.3-8% w/w brivudine.

8. A pharmaceutical composition according to claim 7, containing 0.5-5% w/w brivudine.

9. A pharmaceutical composition according to any preceding claims which is in the form of (O/W or W/O) cream, lipstick, lipogel, hydrogel.

10. A pharmaceutical composition according to claim 9, which is selected from the group consisting of:
i) O/W cream containing Brivudine 1%, Titanium dioxide 25%, Aluminium oxide 1.6%, Silica 0.1%, Glycerol 0.4%, Simethicone 0.5%, Steric acid 1.9%, Propylene glycol 15%, Vasilinum album 9%, Polyoxyethylenemonostearate 2%, Cetostearyl alcohol 1.5%, Citric acid (20%) 0.6%, 4-Methylhydroxybenzoate 0.15%, Benzalkonium chloride 0.2%, Polydimethylsiloxane 0.2%, Water ad. 100%
ii) lipstick formulation containing Brivudine 5%, Titanium dioxide 20%, Glycerol monostearate 10%, Glycerol ester of fatty acids C10-C18 20%, Propylene glycol 15%, Caprylic acid triglycerid, Capric acid triglycerid 15%, Cera alba 4%, Ester of diglycerol and caprylic-, capric-, isostearate-, adipic acid 11%
iii) lipogel containing Brivudine 5%, Paraffinum perliquidum 34%, Isopropyl myristate 25%, Silica 6.25%, Sorbitan Monooleate 3%, Titanium dioxide 16.25%, Aluminum oxide 5.125%, Iron oxide yellow 2.1%, Iron oxide red 0.6%, Iron oxide black 0.3%, Glycerol 2.375%
iv) W/O-emulsion containing Brivudine 0.5%, Zink oxide. 10%, Titanium dioxide 10%, Paraffinum subliquidum 15% Propylene glycol 8%, Vasilinum album 6%, Lanolin 5%, Sorbitan monooleate 3%, Cera alba 1.5%, Zinc stearate 1.0%, Magnesium stearate 1.0%, 4-Methylhydroxybenzoate 0.15%, Benzalkonium chloride 0.2%, Propyl gallate 0.02%, Citric acid (20%) 0.6%, Water ad. 100%
v) hydrogel containing Brivudine 5%, Titanium dioxide 21.25%, Aluminum oxide 1.25%, Silica 0.125%, Glycerol 2.375%, Iron oxide yellow 3.25%, Iron oxide red 1.25%, Iron oxide black 0.5%, Propylene glycol 20%, Paraffinum subliquidum 5%, Isopropyl myristate 5%, Cetyl alcohol 3%, Polyoxyethylenemonostearate 0.8%, Hydroxyethylcellulose 0.3%, Citric acid q. s., Water ad. 100%
vi) W/O-emulsion containing Brivudine 5%, Titanium dioxide 22.5%, Paraffinum subliquidum 15%, Propylene glycol 10.5%, Vasilinum album 6%, Lanolin 5%, Sorbitan monooleate 3%, Cera alba 1.5%, Zinc stearate 1%, Magnesium stearate 1%, 4-Methylhydroxybenzoate 0.15%, Benzalkonium chloride 0.2%, Propyl gallate 0.02%, Water ad. 100%
vii) hydrogel containing Brivudine 1%, Titanium dioxide 27%, Propylene glycol 15%, Paraffinum subliquidum 5%, Isopropyl myristate 5%, Cetyl alcohol 3%, Polyoxy-ethylenemonostearate 0.8%, Hydroxyethylcellulose 0.3%, 4-Methylhydroxy-benzoate 0.15%, Benzalkonium chloride 0.2%, Citric acid q. s., Water ad.100%
viii) lipogel containing Brivudine 2%, Iron oxide yellow 0.75%, Iron oxide red 0.15%, Iron oxide black 0.1%, Isopropyl myristate 24%, Titanium dioxide 15%, Paraffinum subliquidum 15%, Propylene glycol 15%, Vasilinum album 12%, Zinc oxide 5%, Silica 5%, Sorbitan trioleate 3%, Cera alba 3%
ix) O/W cream containing Brivudine 5%, Zinc oxide 10%, Titanium dioxide 10%, Aluminium oxide 0.6%, Silica 0.032, Glycerol 0.1%, Iron oxide yellow 2.6%, Iron oxide red 1%, Iron oxide black 0.4%, Propylene glycol 20%, Vasilinum album 10%, Paraffin oil, subliqu. 6%, Polyoxyethylene-monostearate 3%, Cetostearyl alcohol 2%, Citric acid (20%) 0.6%, Polydimethylsiloxane 0.5%, Water ad. 100
x) O/W cream containing Brivudine 2%, Titanium dioxide 21.27%, Aluminium oxide 1.38%, Silica 0.07%, Glycerol 0.28%, Iron oxide yellow 1.4%, Iron oxide red 0.4%, Iron oxide black 0.2%, Propylene glycol 20%, Vasilinum album 9%, Paraffin oil, subliqu. 5%, Polyoxyethylenemonostearate 3%, Cetostearyl alcohol 2%, Citric acid (20%) 0.8%, Polydimethylsiloxane 0.3%, Water ad. 100%

11. A pharmaceutical composition according to any preceding claims, for the topical treatment of varicella zoster virus and herpes simplex type 1 virus infections.

12. The use of metal-oxide pigments as photo-stabilizer for brivudine topical formulations.

13. The use according to claim 12 of one or more pigments selected from the group consisting of titanium dioxide, iron oxide, zinc oxide.
